(19) United States
(12) Patent Application Publication
(10) Pub. No.: US 2013/0172725 A1
(43) Pub. Date: Jul. 4, 2013

(54) BIOMETRIC INFORMATION SENSING DEVICE

(71) Applicant: Chih-Liang Wu, Taoyuan County (TW)

(72) Inventor: Chih-Liang Wu, Taoyuan County (TW)

(21) Appl. No.: 13/705,916

(22) Filed: Dec. 5, 2012

(30) Foreign Application Priority Data
Dec. 30, 2011 (TW) 100150005

(51) Int. Cl. A61B 5/00

(52) U.S. Cl. CPC A61B 5/6833
USPC 600/391

(57) ABSTRACT

A biometric information sensing device for attaching to a body surface of a biological body for sensing the biometric information such as heartbeat, respiration, positioning, or body movement includes at least one piezoelectric layer having piezoelectric conversion function. The piezoelectric layer includes at least one bending portion and at least one adhesive portion, wherein the adhesive portion is directly or indirectly attached to the biological body to sense deformation of the surface of the biological body and the bending portion enhances the deformation to transform into an electrical sensing signal to transmit to a circuit broad, via signal processing and transmits to a remote control unit for analyzing the biometric information of the biological body. The sensitivity is enhanced by the bending portion of the piezoelectric layer to improve signal-to-noise ratio.

### BACKGROUND OF THE INVENTION

1. Field of the Invention

The present invention relates to a biometric information sensing device, and more particularly to a biometric information sensing device including a piezoelectric layer with a bending portion to enhance sensitivity of physiological information of a living creature and to improve signal-to-noise ratio.

2. The Related Art

Recently, due to growth of aging gradually, westernized food and other factors results in chronic disease and hence the sick population grows, which, in turn, exceeds a load burden of the general society or community can bear. Cardiovascular diseases, such as heart disease, diabetes, high blood pressure, high cholesterol and etc., are chronic diseases with very high prevalence in the developed countries. In normal circumstances, a lot of people suffering from these illnesses usually have no obvious symptoms. However, patients with cardiovascular disease are the risky groups of sudden cardiac death. Once the condition suddenly deteriorated, the patient will lose the pulse and consciousness within a few seconds, and then lose the ability to breathe autonomously. If no urgent medical intervention is provided to him within ten minutes, it can result in a high fatality.

Generally, a majority of elderly people and those people with restricted activity and poor action may pass away due to sudden cardiac attack, owing to the difficulties for them to call for assistance at the onset of a disease within a few seconds. What worse is, according to statistics, more than half of sudden cardiac death occurs under the circumstance without any person nearby, hence resulting in the difficulty of calling for urgent assistance. According to statistics, in many developed countries, sudden cardiac death has been ranked one of the leading causes of death for many years. Therefore, in order to monitor the deteriorating situation of patients with cardiovascular disease, a demand for relevant biometric information monitoring system is gradually increasing.

Moreover, in addition to the group of sudden cardiac death, sudden infant death syndrome (SIDS) is also one of the widely spotted acute fatal diseases. According to statistics of the U.S. National Institutes of Health, 1/2000 newborn infants died of sudden infant death syndrome each year. Basically, in the process of this disease, the infants may lose their vital signs, such as heartbeat and respiration. In general, the occurrence of sudden infant death syndrome is quite unpredictable, besides infants are less capable of calling for help, these phenomena lead to difficulties in medical intervention and urgent treatment in time of need. In addition, in the home environment, the elderly people or infants may choke while eating or air blocked while sleeping and hence results in asphyxia and death. These news are heard from time to time.

During the process of sudden cardiac arrest, sudden infant death syndrome and sign of asphyxiation may cause patients heart or respiratory arresting, so that if we can discover such patients immediately who lose their vital signs due to diseases or accident events, we can grasp the best opportunity to give urgent treatment and send them to the hospital.

Patients in the hospital have a perfect vital sign monitoring system, which conducts vital sign monitoring immediately and continuously, but it is limited by the volume thereof which is too large or wire is constricted and so on, it usually results in the monitoring action been confined in the ward. In addition, in the general elder nursing home or the home environment, due to the disadvantages like monitoring equipment is expensive and poor portability, the equipment like vital sign monitoring is still not prevalent to the general environment.

To this end, some industries have developed some portable physiological sensing devices, capable of sensing a variety of biometric information, such as respiration or heartbeat, may sense abnormalities and send alarm at the early stage when the patients stop their vital signs, whereby enabling the concerned people to provide urgent treatment. However, the currently existing portable physiological sensing devices have many disadvantages like the volume thereof is too large, poor portability and unreliable sensing signals.

Referring to FIG. 1, a schematic diagram of a respiration and heartbeat sensing device of the prior art is shown. The respiration and heartbeat sensing device includes a substrate A and a piezoelectric layer B, wherein the substrate A is attached to the human body, such as the chest, the piezoelectric layer B is arranged on the substrate A, and the piezoelectric layer B can transform the deformation into electric signal by piezoelectric transform operation, where the deformation is generated by the piezoelectric layer B due to respiration or heartbeat of human body in term of information. The information includes respiration and heartbeat such as the frequency and amplitude of the respiration and heartbeat. Moreover, by using electronic device (not shown) to capture information contained in the electrical signal, and continuously record so as to determine whether the respiration or heartbeat is normal, and warn the operator in the event of an abnormal status, such as the warning light or warning bell is turned on, or transmitting a warning message to the families, medical staff, nursing staff. Therefore, obtaining the records of respiration or heartbeat as a reference data of physiological status, can save a lot of manpower and improve efficiency of urgent treatment.

In addition, by using wireless transmitting components, the electrical signal is transmitted to a remote computer or server, whereby establishing remote monitoring function to expand coverage, which is very suitable for large hospitals, nursing homes and special training base.

However, the disadvantages of the prior art is in that the piezoelectric layer is a flat shape, is unable to generate large deformation due to respiration or heartbeat, such that the transformed electrical signal is too weak, particularly in the interference from other noise which is unable to sense the respiration or heartbeat correctly. Therefore a biometric information sensing device of the piezoelectric layer using the bending configuration of the piezoelectric layer to improve the sensing sensitivity and to increase the strength of the electrical signal is required, which can solve the conventional technical problems mentioned above.

### SUMMARY OF THE INVENTION

The purpose of the present invention is to provide a biometric information sensing device, which is adapted to be attached to a body surface of a biological body in order to sense the biometric information, such as respiration and heartbeat. The biometric information sensing device includes at least one piezoelectric layer having piezoelectric conversion function. The piezoelectric layer includes at least a bending portion and an adhesive portion, wherein the adhesive portion is attached to the biological body to sense deformation of the surface of the biological body, and the deformation is enhanced by the bending portion and is transformed thereof into an electric sensing signal and then is transmitted to a circuit board, to a remote control unit via signal processing for analyzing the biometric information.

In addition, the biometric information sensing device of the present invention further includes a substrate, a connection layer or a supporting layer arranged onto a surface of the piezoelectric layer to increase the sensing ability of the piezoelectric layer and is facile to attach to the biological body. It can greatly enhance the sensitivity of the device and improve signal-to-noise ratio of the biometric information.

Accordingly, the biometric information sensing device of the present invention is suitable to assist the doctors or the medical staff monitoring the physiological status of patients immediately. When their heartbeat or respiration status is abnormal, such as asphyxia, shock, fainting, ventricular fibrillation, arrhythmias or cardioplegia, the device can remind the doctors or the medical staff to proceed necessary treatment to prevent the accidents. Moreover, the biometric information sensing device of the present invention can record the physiological status of the biological body in a long time to determine the healthy status or reference of clinical diagnosis to save human resource and improve the efficiency of the required treatment.

### BRIEF DESCRIPTION OF THE DRAWINGS

FIG. 1 is a schematic drawing of a respiration and heartbeat sensing device of the prior art.

FIG. 2 is a schematic drawing of a biometric information sensing device of the present invention.

FIG. 3 is a schematic drawing of shapes of embodiments of a piezoelectric layer employed in the biometric information sensing device of the present invention.

FIG. 4 is another schematic drawing of the biometric information sensing device of the present invention.

FIG. 5 is a block diagram of a circuit board of the biometric information sensing device of the present invention.

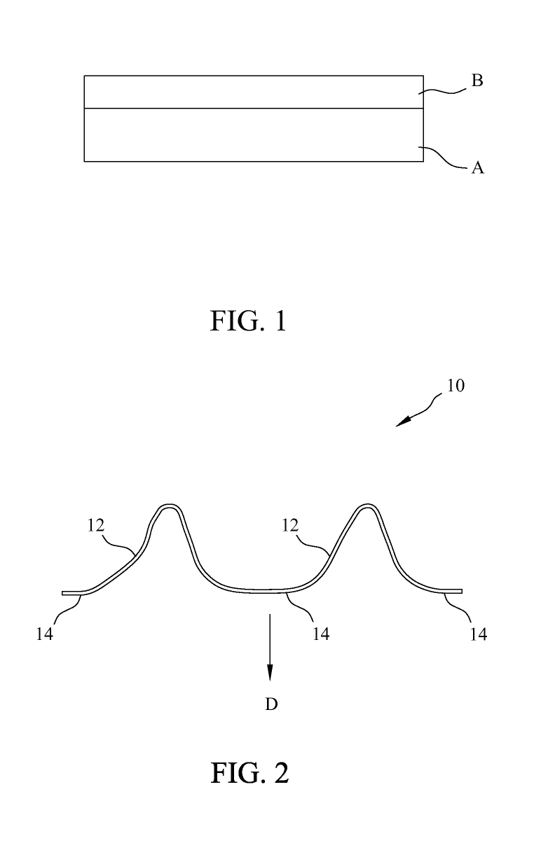

### DETAILED DESCRIPTION OF THE EMBODIMENTS

The present invention is further described by figures and the reference numbers as follows.

Referring to FIG. 2, the schematic drawing of a biometric information sensing device of the present invention is shown. The biometric information sensing device of the present invention includes: at least one piezoelectric layer 10 having piezoelectric conversion function, only one piezoelectric layer 10 is shown in FIG. 2, each piezoelectric layer 10 includes at least one bending portion 12 and at least one adhesive portion 14, wherein the adhesive portion 14 is adapted to be attached to the biological body (not shown) as direction D shown in FIG. 2 in order to sense deformation of the surface of the biological body, where the deformation is caused by physiological operation, such as respiration or heartbeat. The bending portion enhances sensing the deformation. Thus, the piezoelectric layer 10 can transform the sensed deformation into an electric sensing signal.

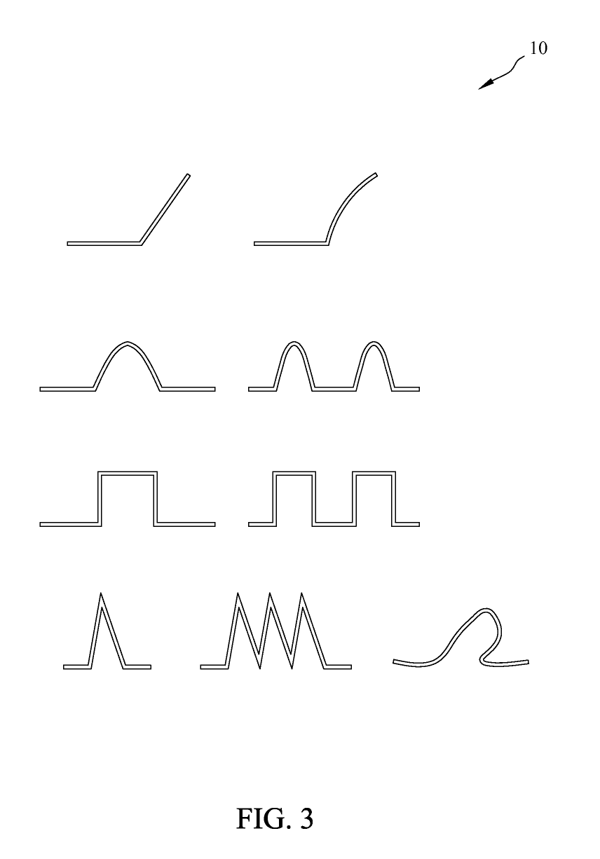

It is noted, the shape of the piezoelectric layer 10 shown in FIG. 2 is an exemplary to easy explain the characteristics of the present invention, which is not to limit the scope of the present invention. Thus, the shapes of the bending portion 12 can be inclined, arched, stair convex, triangle, serrate or irregular bending, as best shown in FIG. 3.

The piezoelectric layer 10 is made from stiffness or flexible materials, such as at least one of quartz, silicon dioxide (SiO₂), lithium niobate (LiNbO₃), lithium tantalate (LiTaO₃), barium titanate (BaTiO₃), lead zirconate titanate (Pb(Zr,Ti)O₃), gallium arsenide (GaAs), aluminum nitride (AlN), zinc oxide (ZnO), bismuth ferrite (BiFeO₃), polyvinylidene fluoride (PVDF), polyvinyl chloride (PVC), polyethylene terephthalate (PET), polyethylene (PE), polypropylene (PP), polyvinyl chloride (PVC), polystyrene (PS), polytetrafluoroethene (PTFE), polymethylmethacrylate (PMMA) and polydimethylsiloxane (PDMS).

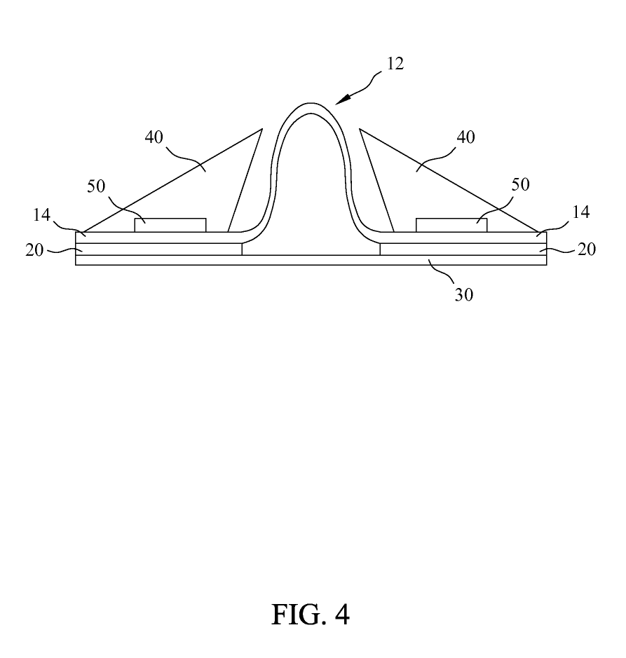

The biometric information sensing device of the present invention further includes at least one connection layer 20, a substrate 30, at least one supporting layer 40 or a circuit broad 50, as shown in FIG. 4. It is noted, FIG. 4 is merely a exemplar, thus, the arrangement of the connection layer 20, the substrate 30, the supporting layer 40 or the circuit board 50 which is capable of performing the function of the present invention may belong to the scope of the present invention, for example, partly or entirely arranged below or above the bending portion 12 or adhesive portion 14 of the piezoelectric layer 10.

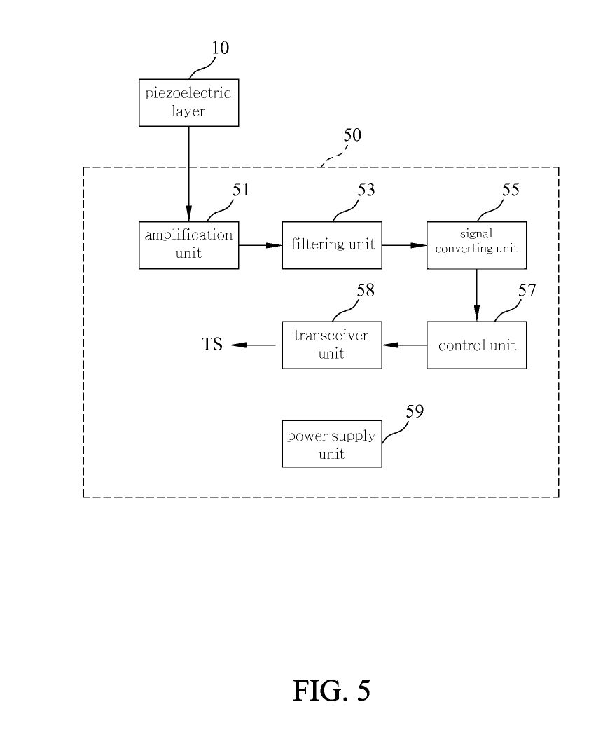

The electric signal processing of the biometric information sensing device of the present invention is performed by the circuit board 50. Referring to FIG. 5, the function block diagram of the circuit board of the biometric information sensing device of the present invention is shown. The circuit board 50 is connected electrically to the piezoelectric layer 10 to receive the sensing signal generated by the piezoelectric layer 10, in form of wired signal or wireless signal and is transmitted to a remote control unit (not visible).

The circuit board 50 includes at least one amplification unit 51, at least a filtering unit 53, at least a signal converting unit 55, at least a control unit 57, at least a transceiver unit 58 and at least a power supply unit 59. The power supply unit 59 is a battery for supplying power to the amplification unit 51, the filtering unit 53, the signal converting unit 55, the control unit 57 and the transceiver unit 58. The amplification unit 51 receives and amplifies the sensing signal and filters noise through the filtering unit 53 while the signal converting unit 55 converts the analog signal to the digital signal, the control unit 57 generates a transmitting signal, which is received by the transceiver unit 58 that generates the wired signal or the wireless signal and further transmits the same.

A partial or an entire of the amplification unit 51, filtering unit 53, signal converting unit 55, control unit 57 and transceiver unit 58 is formed by separated circuit element to simplify the complexity of electric design and alternately, is formed by using integrated circuit (IC) and hence the entire volume of the device is greatly reduced to be portable, particularly in way of patch or applique to form the biometric information sensing device of the present invention so as to monitor or record the biometric information of respiration or heartbeat of the biological body for a long time. Moreover, the remote control unit can be a remote computer or server.

The supporting layer 40 covers or contacts the piezoelectric layer 10 to enhance the stiffness of the biometric information sensing device. The supporting layer 40 is made from at least one of metal, glass, food grade silicone, polyethylene terephthalate (PET), polyethylene (PE), polypropylene (PP), polyvinyl chloride (PVC), polystyrene (PS), polytetrafluoroethene (PTFE), polymethylmethacrylate (PMMA) and polydimethylsiloxane (PDMS)

The connection layer 20 is arranged onto a part of surface of the piezoelectric layer 10 and/or supporting layer 40 for further enhance the stiffness of the biometric information sensing device to improve the sensitivity thereof. In addition, the connection layer 20 is made from glass, polyethylene terephthalate (PET), polyethylene (PE), polypropylene (PP), polyvinyl chloride (PVC), polystyrene (PS), polytetrafluoroethene (PTFE), polymethylmethacrylate (PMMA) and polydimethylsiloxane (PDMS).

The characteristic of the present invention is: the biometric information sensing device can be easily attached to the biological body, such as the chest of the human body, close to the relative portion of heart or lung. Therefore, when the biological body is breathing or heart is beating, the piezoelectric layer 10 of the biometric information sensing device can sense the vibration of the biological body, or sense the respiration or heartbeat of the biological body to generate sensing signal, particularly the bending portion 12 of the piezoelectric layer 10 can generate greater value of the deformation when the surface of the biological body moves laterally or longitudinally, thus enhances the intensity of the sensing signal and improve signal-to-noise ratio.

The sensing signal of the piezoelectric layer 10 includes biometric information relative to respiration and heartbeat of the biological body, such as frequency and amplitude of the respiration and heartbeat. Accordingly, after the remote control unit receives wired signal or wireless signal TS from transceiver unit 58, it can capture the biometric information relative to the heartbeat or respiration to determine whether the function of respiration or heartbeat of the biological body is normal or not, and can generate alarm message when an abnormal and emergent status is occurred so as to inform the medical staff concerned to proceed necessary and urgent treatment to prevent accidents, like asphyxia, shock, fainting, ventricular fibrillation, arrhythmias or cardioplegia.

While the invention has been described in connection with what is considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A biometric information sensing device for attaching to a biological body in order to sense biometric information of the biological body, comprising:
at least one piezoelectric layer having piezoelectric conversion function, the piezoelectric layer including at least one bending portion and at least one adhesive portion, the adhesive portion being directly or indirectly attached to the biological body for sensing deformation of a surface of the biological body and converting into a sensing signal.

2. The biometric information sensing device of claim 1, wherein the piezoelectric layer consists of at least one of quartz, silicon dioxide (SiO₂), lithium niobate (LiNbO₃), lithium tantalate (LiTaO₃), barium titanate (BaTiO₃), lead zirconate titanate (Pb(Zr,Ti)O₃), gallium arsenide (GaAs), aluminum nitride (AlN), zinc oxide (ZnO), bismuth ferrite (BiFeO₃), polyvinylidene fluoride (PVDF), polyvinyl chloride (PVC), polyethylene terephthalate (PET), polyethylene (PE), polypropylene (PP), polyvinyl chloride (PVC), polystyrene (PS), polytetrafluoroethene (PTFE), polymethylmethacrylate (PMMA) and polydimethylsiloxane (PDMS).

3. The biometric information sensing device of claim 1, further comprising at least one circuit board connected electrically to the piezoelectric layer for receiving the sensing signal and generating wired signals or wireless signals to transmit to a remote control unit, the circuit board including at least an amplification unit, at least a filtering unit, at least a signal converting unit, at least a control unit, at least a transceiver unit and at least a power supply unit, the power supply unit supplying power to the amplification unit, the filtering unit, the signal converting unit, the control unit and the transceiver, the amplification unit receiving and amplifying the sensing signal while the filtering unit filters noise, the signal converting unit converts an analog-to-digital signal and the control unit generates a transmitting signal, which is received by the transceiver unit and which generates the wired signal or the wireless signal.

4. The biometric information sensing device of claim 3, wherein each of the amplification unit, the filtering unit, the signal converting unit, the control unit and the transceiver unit is formed by a separated circuit element, or partly or entirely formed by an integrated circuit (IC).

5. The biometric information sensing device of claim 1, further comprising at least one supporting layer, the supporting layer covering or contacting the piezoelectric layer for enhancing stiffness of the biometric information sensing device, wherein the supporting layer consists of at least one of metal, glass, food grade silicone, polyethylene terephthalate (PET), polyethylene (PE), polypropylene (PP), polyvinyl chloride (PVC), polystyrene (PS), polytetrafluoroethene (PTFE), polymethylmethacrylate (PMMA) and polydimethylsiloxane (PDMS).

6. The biometric information sensing device of claim 5, further comprising at least one connection layer for enhancing the rigidity of the biometric information sensing device, which is arranged onto a part of the surface of the piezoelectric layer and/or the supporting layer, the connection layer consists of at least one of glass, polyethylene terephthalate (PET), polyethylene (PE), polypropylene (PP), polyvinyl chloride (PVC), polystyrene (PS), polytetrafluoroethene (PTFE), polymethylmethacrylate (PMMA) and polydimethylsiloxane (PDMS).
